(19) United States
(12) Patent Application Publication
(10) Pub. No.: US 2006/0009496 A1
(43) Pub. Date: Jan. 12, 2006

(54) Method for preventing hemoprotein and heme-mediated lipid peroxidation

(76) Inventors: John A. Oates, Nashville, TN (US); Lyman Jackson (Jack) Roberts II, Gallatin, TN (US); Olivier Gilles Jean-Noel Boutaud, Nashville, TN (US)

Correspondence Address: DONNA J. RUSSELL, 1492 ANTHONY WAY, MT. JULIET, TN, 37122, US

(21) Appl. No.: 11/153,134

(22) Filed: Jun. 15, 2005

Related U.S. Application Data
(60) Provisional application No. 60/579,923, filed on Jun. 15, 2004.
(60) Provisional application No. 60/622,439, filed on Oct. 27, 2004.

(51) Int. Cl. A61K 31/4415

(52) U.S. Cl. 514/345

(57) ABSTRACT

The invention provides a method for inhibiting hemoprotein- and heme-induced lipid peroxidation, a method for identifying compositions that are effective to inhibit hemoprotein- or heme-induced lipid peroxidation, and methods of therapeutic use for those compounds.

### CROSS-REFERENCE TO RELATED APPLICATIONS

This application claims the benefit of earlier-filed U.S. provisional application No. 60/579,923, filed Jun. 15, 2004, and 60/622,439, filed Oct. 27, 2004, and the disclosures both are incorporated herein by reference.

### Statement in Regard to Government Rights

This invention was made in part with funding provided by the United States Government Grant numbers GM 15431 and 42056, awarded by the National Institutes of Health. The U.S. Government may therefore have certain rights in this invention.

### FIELD OF THE INVENTION

The present invention relates to methods for inhibiting the formation of hemoprotein- and heme-mediated oxidation products and methods for preventing tissue and organ damage associated with hemoprotein- and heme-mediated oxidation and oxidation products.

### BACKGROUND OF THE INVENTION

Oxidative stress has been associated with a number of disease states, including cardiovascular disorders, neurological disorders, cancer, and diabetes. Iron and free heme have also been associated with cardiovascular disorders, neurological disorders, cancer, and diabetes. Heme acts as a pro-inflammatory molecule involved in the pathology of conditions as diverse as renal failure, arteriosclerosis, and peritoneal endometriosis.

Although correlations have been found between lipid peroxidation and a wide variety of seemingly diverse diseases, and certain oxidized lipids have also been proposed as markers which indicate the presence of or level of oxidative damage, it would be of great value to identify the biochemical processes that produce such oxidative damage and to identify pharmaceutical agents that may prevent it.

### SUMMARY OF THE INVENTION

The present invention provides a method for inhibiting, hemoprotein and heme-mediated lipid peroxidation in a mammalian subject. In one embodiment, the method comprises administering a therapeutically effective amount of an agent that blocks the hemoprotein and heme-mediated oxidation of arachidonic acid to form isoprostanes. In various embodiments, this may be accomplished by providing a therapeutically effective dosage of acetaminophen, an acetaminophen derivative or an acetaminophen analog to prevent or treat hemoprotein and heme-mediated oxidation. It may also be accomplished by providing an effective amount of a Vitamin E homolog that is effective as an inhibitor of hemoprotein and heme-mediated formation of isoprostanes.

The invention also provides a method for preventing tissue damage associated with heme, which may be present when hemoglobin, myoglobin, or other heme-containing agents are released from damaged tissues. The invention provides a therapeutic method for ameliorating the effects of hemoprotein and heme-mediated lipid peroxidation products on the kidney, and provides a method for preventing or treating acute kidney failure following tissue injury resulting in release of hemoglobin or myoglobin from the intracellular environment.

The invention also provides a method for preventing or decreasing tissue damage associated with lipid peroxidation and lipid peroxidation products such as isoprostanes. In one embodiment, the invention relates to a method for preventing acute renal failure in a mammal by administering at least one thromboxane receptor antagonist to an individual who is at risk for the development of rhabdomyolysis-induced renal failure or the hepatorenal syndrome.

The invention also provides a method for treating acute renal failure in a mammal by administering at least one thromboxane receptor antagonist to an individual who has demonstrated symptoms of acute renal failure or has been diagnosed with acute renal failure.

In one embodiment of the invention, the thromboxane receptor antagonist is a compound that is a derivative of the formula described in U.S. Pat. No. 5,128,359:

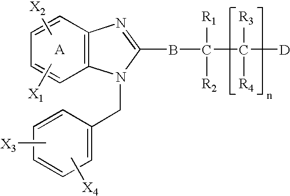

In another embodiment, the TxR antagonist is 7-[3-[[2-[(phenylamino)carbonyl]hydrazino]methyl]7-oxabicyclo[2.2.1]hept-2-yl]-[1S-[1 alpha,2 alpha(Z),3 alpha,4 alpha]]-]5-heptenoic acid (SQ29548, Bristol-Myers Squibb).

The invention provides a method for identifying agents that are effective to inhibit hemoprotein and heme-mediated lipid peroxidation.

### BRIEF DESCRIPTION OF THE DRAWINGS

FIG. 1 is a graph illustrating the correlation between severity of disease in Falciparum malaria, as indicated by serum lactate levels, and urinary F₂-isoprostanes levels.

FIG. 2 is a bar graph illustrating the effectiveness of adminstration of a thromboxane receptor antagonist in improving renal function, as measured by creatinine clearance, in rats with rhabdomyolysis. Creatine clearance is shown on the Y axis, and the X axis is labeled to indicate the identity of the control group, the group with rhabdomyolysis who did not receive TxR antagonist therapy, and the group with rhabdomyolysis who received TxR antagonist therapy. Each group consisted of about 6 rats.

FIG. 3 illustrates effects of acetaminophen administration on creatinine clearance (top graph) and plasma creatinine (bottom graph) in rhabdomyolysis. Rhabdo/Ac indicates subjects with rhabdomyolysis treated with acetaminophen. Rhabdo/Sal indicates subjects with rhabdomyolysis treated with saline. Creatinine clearance is expressed in milliliters per minute (Y axis), and plasma creatinine levels are expressed as μmoles per liter (Y axis).

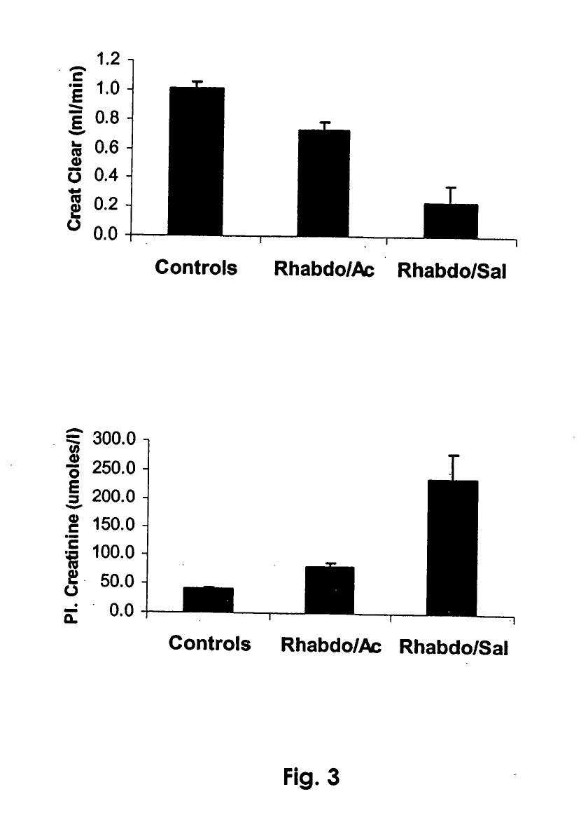

FIG. 4 is a graph illustrating the inhibitory effects of acetaminophen, Trolox (Analog A), 2-dimethylamino-5-hydroxypyridine (DM-Pyr) and 2-dimethylamino-5-hydroxypyrimidine (DM-Pym) on oxidation of arachidonic acid in the presence of hemoglobin and 150 μM hydrogen peroxide.

### DETAILED DESCRIPTION

The inventors have discovered that lipid peroxidation products, including isoprostanes, which are products of hemoprotein and heme-mediated oxidation of arachidonic acid, are responsible for many of the damaging effects associated with the release of hemoglobin or myoglobin from damaged tissues, and that inhibiting formation of these oxidation products can provide a therapeutic benefit for prevention or treatment of a variety of conditions associated with hemoprotein and heme-mediated tissue damage. This can be accomplished by inhibiting the formation of the oxidation products themselves, or interfering with their action once oxidation products are formed.

The inventors have discovered a method for identifying therapeutic agents that can inhibit hemoprotein and heme-mediated lipid peroxiddtion.

F₂ isoprostanes are formed by free radical peroxidation of esterified arachidonic acid. Isoprostanes are considered to be a reliable index of lipid peroxidation in vitro, as they are formed completely in situ in phospholipids and are subsequently released by phospholipase. Studies indicate that measuring isoprostanes (IsoPs) in body fluids provides a reliable method for assessing oxidative stress in animals and humans. The inventors have demonstrated that, in addition to being oxidative stress markers, isoprostanes are also causative agents of organ and/or tissue damage that can be preventing or treated using inhibitors of isoprostane synthesis resulting from arachidonic acid oxidation. Isoprostanes are present in the nanomolar range in normal human plasma and increase up to 100- to 200-fold during periods of oxidative stress, which characterize a variety of disease states. Physiological responses to isoprostanes can be observed in the submicromolar range. Concentrations of isoprostane 8-iso-PGF_{2α} in the range of 1 nmol/L to 1 μmol/L have previously been shown to induce potent vasoconstriction, platelet shape change, increased inositol phosphate levels, calcium release from intracellular stores, platelet adhesion and platelet aggregation. The inventors have demonstrated that isoprostanes produce physical and functional damage to organs through their actions, and that this damage can be prevented or treated by administration of pharmaceutical agents that inhibit heme oxidation-mediated production of isoprostanes.

Circulating levels of F₂-isoprostanes have been shown to increase by as much as 200-fold in animal models of oxidant injury. 8-epi-PGF_{2d}, an F₂-isoprostane, has been found to be one of the most potent renal vasoconstricting substances ever identified (Morrow, et al., Proc. Nat. Acad. Sci. (1990) 87: 9383-9387), and the renovascular effects of 8-epiPGF2 have been shown to result from activation of thromboxane receptors (Takahashi, et al. J. Clin. Invest. (1992) 90: 136-141). In rat renal circulation, for example, the constrictor effects of 8-epi-PGF_{2α} are caused by its activation of thromboxane receptors.

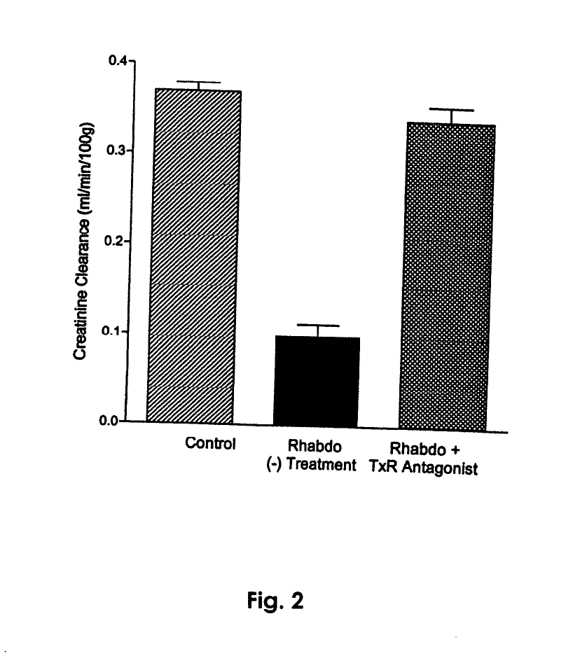

The inventors discovered a correlation between increased urinary isoprostanes and decreased kidney function in rhabdomyolysis-induced renal failure, hepatorenal syndrome, and Falciparum malaria, and proposed that if isoprostanes were a causative agent of kidney damage thromboxane receptor antagonists might improve kidney function associated with the acute kidney failure associated with these disorders. Furthermore, the inventors suspected that hemoprotein and heme-mediated oxidation might be an important factor in the production of isoprostanes from arachidonic acid. They chose a model of acute kidney damage associated with myoglobin release, rhabdomyolysis, to test their hypothesis. The inventors discovered that thromboxane receptor antagonists, provided to an individual suffering from rhabdomyolysis, can significantly increase kidney function, as shown in FIG. 2. They also determined that thromboxane receptor antagonists, provided to a patient in whom kidney function is reduced due to hepatorenal syndrome, also significantly increase kidney function. The increase in kidney function in both instances is sufficient to return to nearly normal kidney function without additional therapeutic measures.

These results demonstrate that by inhibiting the detrimental effects of isoprostane formation through the use of thromboxane receptor antagonists, kidney function can be restored to almost normal levels in humans with hepatorenal syndrome.

Thromboxane receptor antagonists as used by the inventors in the method of the present invention are described in U.S. Pat. No. 5,128,359 and have a basic structure such as, for example: 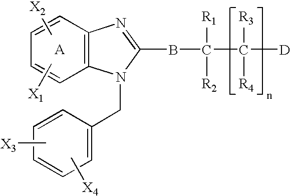
Other compounds classified as thromboxane receptor antagonists, such as the TxR antagonist 7-[3-[[2-[(phenylamino)carbonyl]hydrazino) methyl]7-oxabicyclo[2.2.1]hept-2-yl]-, [1S-[1 alpha,2 alpha (Z),3 alpha,4 alpha]]-]5-heptenoic acid (SQ29548, Bristol-Myers Squibb), BAYu3405 (Bayer AG, Leverkusen, Germany), and Vapiprost (GR32191 B, Glaxo Wellcome) are also effective in the method of the present invention.

The inventors provide here a method for preventing or treating acute renal failure by administering at least one thromboxane receptor antagonist to a mammal that is at risk of, or has been diagnosed with, acute renal failure. Although patients with acute renal failure can be asymptomatic, the condition can be diagnosed by observed elevations of blood urea nitrogen (BUN) and serum creatinine levels. ARF is usually characterized by the acute increase of serum creatinine levels from baseline, which is generally an increase of at least 0.5 milligrams per deciliter (44.2 micromoles per liter). In symptomatic ind ividuals, symptoms can include, for exa mple, anorexia, fatigue, changes in mental status, nausea and vomiting, pruritis, seizures (associated with high BUN), and shortness of breath.

Therapeutic dosages of thromboxane receptor antagonists for use in the method of the present invention may be determined by those of skill in the art. For example, dosages of about 30 to about 100 micrograms per kilogram body weight per day are effective, showing a dose-dependent response, to block thromboxane receptor-mediated platelet aggregation and are therefore appropriate for blocking the effects of isoprostane-mediated renal damage via the thromboxane receptor.

The inventors also discovered that certain agents can be used to inhibit formation of isoprostanes formed through heme oxidation of arachidonic acid, and that inhibition of isoprostane formation provides prevention or reversal of kidney damage, similar to that seen when thromboxane receptor antagonists were administered.

In the method of the present invention, acetaminophen (paracetamol) (4′-hydroxyacetanilide) has been shown to be a potent inhibitor of hemoprotein and heme-mediated lipid peroxidation and to provide a benefit in restoring kidney function in a rat model of acute kidney failure resulting from rhabdomyolysis. Acetaminophen is sold under a variety of trade names, including Tylenol® (McNeil-PPC, Inc.). “Acetaminophen,” as used herein, comprises paracetamol or 4′-hydroacetanilide, and derivatives or analogs thereof that are functionally equivalent to acetaminophen in their ability to reduce heme protein radicals and the elevated oxidation state of heme iron generated by peroxide. Functional equivalence is defined as having from at least about sixty percent to greater than one hundred percent of the activity of acetaminophen in regard to its capacity to reduce heme protein radicals. For example, acetaminophen inhibits the oxidation of arachidonic acid induced by myoglobin (Mb) in the presence of hydrogen peroxide (H₂O₂) at a mean inhibitory concentration (IC₅₀) of less than 3 μM and inhibits the oxidization of arachidonic acid induced by myoglobin without added H₂O₂ at an IC₅₀ of less than 0.6 μM.

Certain Vitamin E analogs, such as Trolox (a water-soluble carboxylic acid derivative of a-tocopherol) produce a similar, although not quite as potent, effect to that produced by acetaminophen on production of hemoprotein and heme-mediated oxidation products of arachidonic acid.

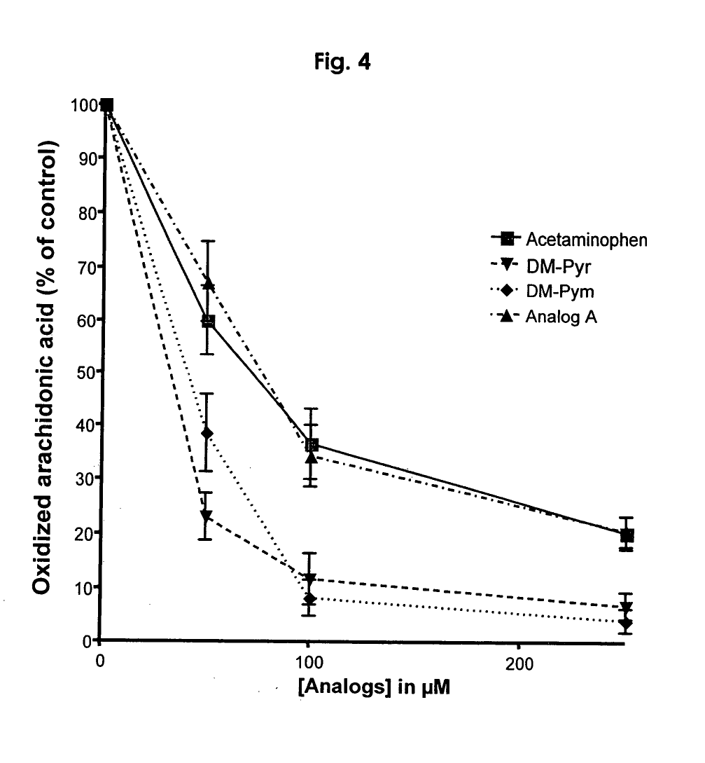

Of even more interest, however, is the fact that a novel class of chain-breaking antioxidants, 6-substituted-2,4-dimethyl-3-pyridinols (Wijtmans, M. et al., J. Org. Chem. 2004, 69: 9215-9223) such as, for example, 2-dimethylamino-5-hydroxypyridine (DM-Pyr) and 2-dimethylamino-5-hydroxypyrimidine (DM-Pym), provide significantly more inhibition of heme-induced arachidonic acid oxidation than does acetaminophen. As seen in FIG. 4, two synthetic compounds demonstrated significantly more potent inhibition of hemoglobin-catalyzed oxidation of arachidonic acid in the presence of hydrogen peroxide than is produced by acetaminophen or Trolox.

This discovery provides an important method for preventing or reducing the effects of heme oxidation and heme oxidation products in the body, and more particularly for preventing or reducing the effects of isoprostane produced from its arachidonic acid precursor. Also provided is a method for treating patients suffering from tissue and/or organ damage associated with heme oxidation or heme-associated lipid peroxidation. Compounds of interest not yet known to inhibit heme-induced arachidonic acid peroxidation may therefore be identified using the method of the present invention. These compounds may provide therapeutic agents for a number of disease conditions in which isoprostanes are the causative agents of tissue damage.

Effective amounts of acetaminophen, an acetaminophen derivative, or an acetaminophen analog (such as an effective Vitamin E homolog or derivative or ester thereof) can be determined by those of skill in the art, given the disclosure of the present invention. Effective amounts reduce heme protein radicals and the elevated oxidation state of heme iron generated by peroxide, thereby reducing oxidation of arachidonic acid to produce isoprostanes and other lipid peroxidation products.

Acetaminophen is available as a non-prescription medication (e.g., Tylenol®), and is most commonly provided in liquid, tablets, caplets, gelcaps, and similar forms. It can be provided with a variety of excipients and other suitable pharmaceutical agents. Acetaminophen can also be administered intravenously.

Therapeutic targets for the method of the present invention therefore include, for example, hemolytic states, rhabdomyolysis, subarachnoid hemorrhage, (hemorrhagic stroke), myocardial reperfusion, periventricular leukomalacia, Falciparum malaria, and Sickle Cell Anemia. The method of the invention is appropriate for use when a condition or disease state is associated with hemolysis or rhabdomyolysis, which result in release of hemoglobin or myoglobin from cells and with heme induced oxidative damage associated with administration of heme-containing or other heme containing products, such as blood substitutes. The method of the invention is especially useful for therapy provided to patients having conditions in which hemoprotein and heme-mediated oxidation would normally, if untreated, result in significant damage. Oxidative damage has been associated with cell death in tissue of the nervous system, kidney, cardiovascular system, and other tissues. Lipid peroxidation damages nerve cells, and vasoconstriction caused by isoprostanes leads to reduced blood flow to tissues.

Subarachnoid hemorrhage, for example, generally occurs when blood is released into the subarachnoid space surrounding the brain and spinal cord. This can be caused by a variety of factors including trauma, ruptured intracranial aneurysm, vasculitis, tumor, anticoagulants, hemophilia, and arteriovenous malformation. Hemoglobin is released, and then becomes oxidized to ferric hemoglobin which is competent to undergo redox cycling to produce free radicals. Subsequently (e.g., 4-5 days after onset of hemorrhage) intense vasoconstriction occurs in the brain, decreasing the nutrients and oxygen available to the neural cells. Furthermore, exposure of neurons to hemoglobin has been associated with cell death in a-dose-dependent manner. Previous research by the inventors has determined that F₂ isoprostanes are produced as a result of hemoprotein and heme-mediated lipid peroxidation and are potent vasoconstrictors. Basu, et al., (FEBS Lett. 2000 Mar. 470: 1-6) concluded that isoprostanes are reliable biomarkers for free radical-catalyzed oxidative brain injury and reperfusion injury after cardiac arrest, for example. Administration of acetaminophen, as a prophylactic or therapeutic measure, to a patient at risk for or suffering from a subarachnoid hemorrhage or other abnormal intracranial bleeding event provides a method for preventing or reducing tissue damage associated with hemoprotein and heme-mediated lipid peroxidation and/or vasoconstriction.

Similarly, periventricular leukomalacia, a condition in which there is bleeding into the ventricle in the brain, can be treated by the method of the present invention to prevent or reduce hemoprotein and heme-mediated oxidation and to protect neurons from damage. Periventricular leukomalacia is seen in premature infants and causes cerebral palsy.

Tissue damage is associated with a number of physiological states caused by disease or trauma, and those tissues often release hemoglobin and/or myoglobin as the result of cellular damage or destruction. Many of these conditions are also associated with acute kidney failure. Rhabdomyolysis, for example, occurs when damaged muscle tissue releases myoglobin. Outside the cell, myoglobin promotes heme-mediated lipid peroxidation, the products of which include isoprostanes, potent vasoconstrictive agents. Lipid peroxidation and vasoconstriction in the kidney contributes to kidney failure, and such kidney failure can be ameliorated by the administration of acetaminophen or an acetaminophen analog in the method of the invention to a patient suffering from rhabdomyolysis.

The present invention provides a method for treating and preventing kidney damage associated with tissue injury such as that seen in rhabdomyolysis. Approximately eight to fifteen percent of patients experiencing acute renal failure do so as a complication of rhabdomyolysis. Rhabdomyolysis occurs when the contents of injured muscle cells leak into the circulation. Myoglobin is carried to the kidneys via the circulation, and approximately thirty percent of individuals with rhabdomyolysis develop acute renal failure. Rhabdomyolysis is associated with both traumatic and non-traumatic illness. These include crush injuries to muscles, gunshot wounds, wasp and hornet stings (particularly when in significant number), diabetic ketoacidosis, seizures, hypothyroidism, heat stroke, severe frostbite, and alcoholism. Rhabdomyolysis has also been associated with the use or abuse of certain drugs such as diphenhydramine, cocaine, 3-hydroxy-3-methylglutaryl-coenzyme A (HMG-CoA) reductase inhibitors (i.e., “statins”), and methylenedioxymethamphetamine (MDMA or “Ecstasy”). Complications of hypothyroidism, for example, include rhabdomyolysis and acute renal failure.

Kidney damage and/or failure is also associated with other disease states in which cellular damage or hemolysis produces heme-associated protein (e.g., myoglobin or hemoglobin) release. These include, for example, cardiac arrest, myocardial infarction and other myocardial ischemic syndromes, surgical trauma, Falciparum malaria, Sickle Cell Disease, and tissue reperfusion.

Acute kidney failure associated with hemolytic disorders occurs when erythrocyte damage results in release of hemoglobin into the system. This can occur in a variety of situations or disease states including, for example, Falciparum malaria, sickle cell disease, HELLP (hemolysis, elevated liver levels and low platelet count) syndrome associated with pregnancy-induced hypertension, acute tubular necrosis, and hemolytic uremic syndrome (HUS). These disorders result in the release of heme (i.e., myoglobin and hemoglobin) into the bloodstream. Heme and its oxidation products are carried to the kidneys through the bloodstream. The kidney has the highest blood supply/g (about 3.5 mL/g/min) of all tissues (about 0.07 mL/g/min for most organs except the lung). Circulating agents are thus delivered at 50 times the rate as for other tissues. Heme proteins cause arachidonic acid and other unsaturated fatty acids to undergo oxidation, with the formation of isoprostanes.

Hemolytic uremic syndrome (HUS) is the most common cause of acute renal failure in the pediatric population, characterized by microangiopathic hemolytic anemia, thrombocytopenia, and acute renal failure. As its name implies, a hallmark of HUS is disseminated hemolysis. HUS also results from infection with a Shiga toxin-producing strain of Escherichia coli, but also is associated with Streptococcus pneumoniae infections and infections with other viruses and bacteria. The method provided by the inventors therefore provides a therapeutic technique for treating and/or preventing renal failure in children and adults with HUS.

The most widely used approach for development of blood substitutes has been the development of hemoglobin-based oxygen carriers (HBOCs). Fluorocarbon-based oxygen carriers (FBOCs) have also been developed, but these have been associated with complement activation, reduced platelet function, and certain other undesirable side-effects. HBOCs are hoped to provide oxygen-carrying agents for prevention of ischemic tissue damage and hypovolemic shock. HBOC development has concentrated on stabilizing the hemoglobin molecule to reduce its potential for oxidation. Heme-containing blood substitutes have, however, been associated with renal toxicity and vasoconstriction. Hemoglobin toxicity has been reported to include interference with normal blood-pressure control mechanisms, gastrointestinal and proinflammatory effects, oxidative stress, pancreatic and liver enzyme changes, and neurotoxicity. In some animal species, it is also associated with the development of heart lesions. When administered in a swine model of severe traumatic shock, diaspirin cross-linked hemoglobin produced severe vasoconstriction, markedly raised blood pressure, reduced cardiac output, and resulted in the death of two out of seven animals (Hess et al., J. Appl Physiol. 1993, 74: 1769-1778). The present invention therefore provides a method for administering a blood substitute in combination with a therapeutically effective amount of an inhibitor of hemoprotein and heme-mediated arachidonic acid oxidation such as, for example, acetaminophen or an acetaminophen analog.

This method provides a preventive and therapy for acute renal failure associated with conditions such as acute tubular necrosis, a major cause of acute renal failure among hospitalized patients, accounting for 38% of acute renal failure in hospitalized patients and 76% of patients in the intensive care unit. Acute tubular necrosis is associated with rhabdomyolysis, hemolysis, sepsis, cirrhosis, heart failure, and radiocontrast nephropathy. In each case, damage to erythrocytes or myocytes, release of heme, and/or production of reactive pigment species increases the oxidative potential in the system to produce isoprostanes.

The method provides a therapy for the treatment of acute (Type I) hepatorenal syndrome, which is characterized by rapidly progressive renal failure with a serum creatinine above 2.5 mg/dl or a glomerular filtration rate (GFR) below 20 ml/min, and which has a 10-week mortality rate approachine 90%, with a median survival of 1.7 weeks.

Acute kidney failure is often associated with pre-eclampsia. Endothelial damage results from the arteriolar spasm that accompanies pregnancy-induced hypertension and pre-eclampsia. Red blood cells become fragmented as they pass through small blood vessels with endothelial damage, resulting in the release of hemoglobin into the blood stream. The method of the present invention provides a therapy for women who are at risk for, or develop, acute renal failure as the result of erythrocyte damage and hemoglobin release.

Approximately 30% of patients who are resuscitated in hospitals during cardiac arrest develop clinically overt signs of acute renal failure. Cardiac arrest produces generalized ischemia, with the release of inflammatory factors and oxidative tissue damage. Inhibition of isoprostane synthesis can be used to prevent or treat acute renal failure in patients experiencing cardiac arrest.

Contrast-induced nephropathy, a complication of cardiac catheterization (e.g., angiography), results in a 15% incidence of acute renal failure. The iodinated contrast media has been reported to promote the intra-renal formation of oxygen free radicals, and N-acetylcysteine, an antioxidant, has demonstrated a potentially protective role. The method of the present invention provides a means to minimize free radical-induced lipid peroxidation and isoprostane formation in individuals undergoing cardiac catheterization utilizing iodinated or other free radical-forming media.

Isoprostane 8-iso PGE2 constricts bronchial vasculature through the activation of thromboxane A-2 selective receptors, which in turn trigger tyrosine kinase and Rho-kinase activities, resulting in powerful vasoconstriction (Tazzeo, T. et al., Br. J. Pharmacol. 2003, 140(4) 759-63.) Heme oxygenase has been associated with protection against airway inflammation and has been reported to be elevated in a number of respiratory disease conditions, such as pulmonary fibrosis and asthma. The present invention provides a method for inhibiting isoprostane production in the lungs and related vasculature, as well as a method for identifying pharmaceutical agents which may prevent isoprostane-mediated constriction of bronchial vasculature and vasoconstriction.

Enhanced urinary excretion of isoprostane 8-iso-PGF(2alpha) has been described in association with both type 1 and type 2 diabetes mellitus, and correlates with impaired glycemic control (Mezzetti, A. et al., Cardiovascular Res. 2000, 47(3): 475-88; Davi, G. et al., Circulation 1999, 99(2): 224-9). Diabetes has also been associated with atherosclerosis and other vascular complications. Davi, et al. demonstrated that F₂-isoprostane 8-iso-prostaglandin (PG)F_{2α}, is enhanced in diabetes mellitus and contributes to platelet activation. Furthermore, in ten out of ten subjects to whom Vitamin E was administered, 8-iso-PGF(2alpha) was significantly decreased. The present invention provides a method for preventing isoprostane formation in diabetic patients, as well as a method for identifying pharmaceutical agents that may provide more potent inhibition of isoprostane formation than does Vitamin E supplementation. A therapeutic regimen employing one or more of these pharmaceutical agents may be used to prevent complications of diabetes and impaired glycemic control.

The method of the present invention provides an important opportunity for therapeutic intervention in a variety of cardiovascular conditions, including cardiomyopathies and coronary heart disease, in which isoprostane levels have been found to be increased. The invention also provides a method for identifying therapeutically effective agents for cardioprotective use.

The invention also provides a method for identifying agents, and agents identified by that method that can be used to prevent hemoprotein and heme-mediated oxidative damage to cells and tissues. The method comprises the steps of (1) incubating the target agent with arachidonic acid, heme, and hydrogen peroxide, and (2) comparing the level of arachidonic acid oxidation to a control that comprises either the absence of an agent or the presence of an agent that is known to lack heme-oxidation inhibiting properties. Heme can be added, for example, in the form of hemoglobin, myoglobin, or a combination thereof. Once a basal level of arachidonic acid is determined under the conditions under which the reaction is performed, a control may not be necessary to provide a direct comparison with each sample. The inventors have demonstrated that isoprostane formation from arachidonic acid is associated with hemoprotein and heme-mediated oxidative tissue damage, and that inhibition of hemoprotein and heme-mediated oxidative damage, or inhibition of the action of the arachidonic acid product formed by hemoprotein and heme-mediated oxidation, is an effective therapeutic method for preventing hemoprotein and heme-mediated oxidative damage.

It is also to be understood that the disclosure provided herein is applicable to mammalian subjects, including human subjects. Lipid peroxidation is associated with disease states in a variety of animals, such as canines, and the compositions and methods of the present invention may therefore have both human and veterinary use. The invention may be further described by means of the following non-limiting examples.

### EXAMPLE 1

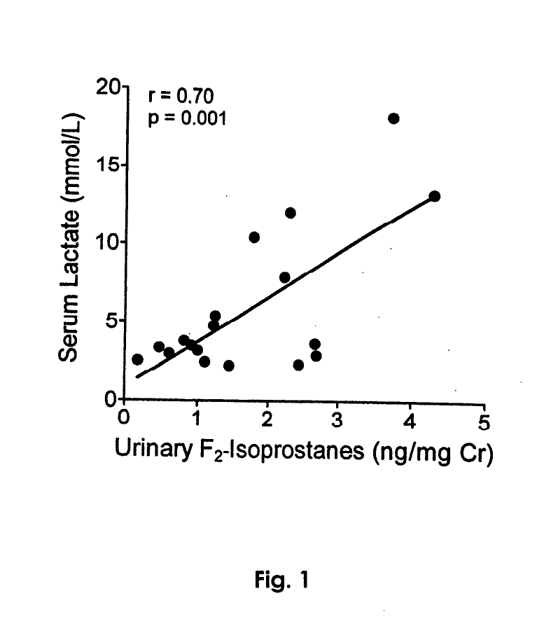

Rats were divided into three groups with approximately six rats in each group. The control group contained rats without rhabdomyolysis, and the two experimental groups were divided into a group with rhabdomyolysis that received treatment with a thromboxone A2 receptor antagonist and a group with rhabdomyolysis that did not receive TxA2 antagonist treatment. Rats with rhabdomyolysis were dosed with BAYu3405 (kindly provided by Bayer AG, Leverkusen, Germany) at a dosage of 10 mg/kg/day. Dosage was administered in TRIS buffer by intraperitoneal (IP) injection. Creatinine clearance was measured by standard methods as rats were followed for 24-48 hours. Results are indicated in FIG. 1, where the level of creatinine clearance is indicated on the Y axis and the experimental/control group is indicated along the X axis. As indicated, kidney function (as measured by creatinine clearance) increased dramatically in the experimental group that received TxR antagonist therapy, as compared to the group that did not. In fact, creatinine clearance in the group receiving treatment returned to levels close to those found in healthy rats.

### EXAMPLE 2

Eighteen subjects were sampled, each having been diagnosed with Falciparum malaria. Serum lactate dehydrogenase levels (a measure of kidney dysfunction) was determined by standard methods, as were levels of urinary F₂-isoprostanes. As indicated in FIG. 2, there is a correlation between increased urinary F₂-isoprostanes and serum lactate levels.

### EXAMPLE 3

Four human subjects, each diagnosed with Type I (acute) hepatorenal syndrome, were provided with a standard dosage of Vapiprost (GR32191B, Glaxo Wellcome). One patient experienced a dramatic increase in kidney function, as measured by standard parameters, 1 patient exhibited no response to therapy, and two patients experienced partial but significant improvement in kidney function.

### EXAMPLE 4

[₁₄C] Arachidonic acid (10 μM) was incubated at 370C for 5 hours with myoglobin (10 μM), hydrogen peroxide (5 μM) and increasing concentrations of acetaminophen. The oxidized species of arachidonic acid were detected by scanning radioactive polar species after separation on thin layer chromatography (TLC). Basal oxidation of arachidonic acid was subtracted from each value at the different concentrations of acetaminophen. Each point shown in FIG. 4 represents the average of two values.

### EXAMPLE 5

Acetaminophen was administered to rats with rhabdomyolysis. Creatinine clearance, a common indicator of kidney function, and plasma creatinine were evaluated by standard methods. As indicated in FIG. 2, creatinine clearance was significantly reduced in animals with rhabdomyolysis after administration of acetaminophen, indicating that acetaminophen administration improves kidney function in rhabdomyolysis.

It is to be understood that the above description is intended to be illustrative, and not restrictive. Many other embodiments will be apparent to those of skill in the art upon reviewing the above description and the above-discussed embodiments may be used in combination with each other.

What is claimed is:

1. A method for inhibiting heme-induced lipid peroxidation in a mammalian subject, the method comprising administering a therapeutically effective amount of an agent that demonstrates effectiveness in blocking hemoprotein or heme-mediated oxidation of arachidonic acid.

2. The method of claim 1 wherein the agent comprises acetaminophen or an acetaminophen analog.

3. The method of claim 1 wherein the agent is chosen from among the group consisting of 2-dimethylamino-5-hydroxypyridine, 2 dimethylamino-5-hydroxypyrimidine, and combinations thereof.

4. A method for preventing or treating isoprostane-mediated tissue damage, the method comprising administering to a subject a therapeutically effective amount of an agent that inhibits heme-induced arachidonic acid oxidation.

5. The method of claim 4 wherein the agent is chosen from the group consisting of acetaminophen, an acetaminophen analog, a Vitamin E homolog, 2-dimethylamino-5-hydroxypyridine, 2 dimethylamino-5-hydroxypyrimidine, and combinations thereof.

6. A method of preventing or treating isoprostane-mediated tissue damage in a mammalian subject comprising administering to the subject a therapeutically effective amount of a thromboxane receptor antagonist.

7. The method of claim 6 wherein the tissue comprises kidney tissue.

8. A method for identifying pharmaceutical agents that reduce isoprostane production, the method comprising the steps of (1) incubating a target agent with arachidonic acid, heme, and hydrogen peroxide, and (2) comparing the oxidization of arachidonic in the presence of the target agent, as compared to a control that comprises the absence of an agent that has heme-oxidation inhibiting properties.

9. The method of claim 8 wherein heme is provided by hemoglobin, myoglobin, or a combination thereof.
